(19) United States
(12) Patent Application Publication
(10) Pub. No.: US 2006/0206995 A1
(43) Pub. Date: Sep. 21, 2006

(54) Shielding device for helmet

(76) Inventors: Wan-Ki Hong, Yongin-si (KR); Bom-Shik Cho, Yongin-si (KR)

Correspondence Address: COLEMAN SUDOL SAPONE, P.C., 714 COLORADO AVENUE, BRIDGE PORT, CT, 06605-1601, US

(21) Appl. No.: 11/329,966

(22) Filed: Jan. 11, 2006

(30) Foreign Application Priority Data
Mar. 16, 2005 (KR) 2005-0021931
Dec. 13, 2005 (KR) 2005-0122485

(51) Int. Cl. A42B 1/08; A61F 9/00

(52) U.S. Cl. 2/424; 2/12

(57) ABSTRACT

A shielding device for a helmet installed so as to secure a sufficient field of vision as well as protect the face portion of the helmet wearer is disclosed. The shielding device for a helmet comprises a shield 10 to cover the opened portion of the helmet body 1; a sun visor 20 which is placed on the inside of the shield 10 to shut off sunlight; a visor 30 which is placed between the shield 10 and the sun visor 20 to block the inflow of rainwater; a hinge joining means 40 which pivotably supports both ends of the shield 10 and sun visor 30 so that they can pivot up and down in one body and/or individually; and a rotation interruption means 50 which obstructs the shield 10, sun visor 20 and visor 30 from descending due to self weight as well as makes the shield 10 be pivoted intermittently with respect to the visor 30.

### BACKGROUND OF THE INVENTION

1. Field of the Invention

The present invention relates to a helmet, and more specifically to a shielding device for a helmet installed so as to secure a sufficient field of vision as well as protect the face portion of the helmet wearer.

2. Description of the Related Art

During the driving of a motorcycle, a helmet is worn for the safety reason of protecting the head of the wearer. And in general, such a helmet is provided with a shielding means for ensuring normal breathing as well as securing the field of vision of the wearer by intercepting wind blowing from front and sunlight.

And, the conventional shield device provided for the helmet is represented by a shield of a transparent or semi-transparent material, and such a shield, in general, is provided with a structure which can be opened or closed up and down. But the conventional shield has a problem of deteriorating the wearing feeling such as making the wearer feel stuffy in hot summertime because it is in such a size as to cover the whole face of the wearer. Meanwhile, there is a helmet which adopts a sun visor in place of such a shield. The sun visor can prevent dazzling of sunlight but cannot shut out wind during driving, so it is not sufficient as a means of shielding.

Therefore, there is a need for a shielding device which can be functioned as a sun visor as well as a conventional shield or in which the function of the sun visor can be optionally used.

### SUMMARY OF THE INVENTION

Therefore, it is an object of the present invention to provide a shield device for a helmet which can enhance the convenience of use by having a plurality of shielding devices comprising a shield, sun visor and visor having different functions such that they are easy to attach and detach and can be optionally used.

In accordance with the present invention, there is provided a shielding device for a helmet comprising: a shield to cover the opened portion of the helmet body; a sun visor which is placed on the inside of the shield to shut off sunlight; a visor which is placed between the shield and the sun visor to block the inflow of rainwater; a hinge joining means which pivotably supports both ends of the shield and sun visor so that they can pivot up and down in one body and/or individually; and a rotation interruption means which obstructs the shield, sun visor and visor from descending due to self weight as well as makes the shield be pivoted intermittently with respect to the visor.

According to a first embodiment of the present invention, the hinge joining means includes a fastening screw which passes through first to third through holes in sequence formed at either end portion of the shield, sun visor and visor.

And, the rotation interruption means includes a first toothed wheel mounted on the inside of either end of the shield, and a second toothed wheel mounted on the outside of either end of the visor.

Preferably, the shielding device further comprises belt-formed sealing members attached on the front of the visor in contact with the inside of the shield.

According to a second embodiment of the present invention, the hinge joining means includes a first screw which passes through in sequence a first through hole formed at either end portion of the shield, a third through hole formed at either end portion of the visor and a second through hole formed at either end portion of the sun visor to screwed to the side of the helmet; and a second screw which passes through a fourth through hole formed at a predetermined distance away from the third through hole of the visor to be fixed on the helmet body.

And, the rotation interruption means includes a first protuberance formed at either end of the shield, a second protuberance formed at either end of the sun visor, and, a third protuberance around the third through hole 31 of either end portion of the visor which is inserted into a slot provided on the first adjacent protuberances 12 and the second adjacent protuberances 22, respectively to support the rotation of these.

Preferably, the third protuberance of the visor has elastic force to obstruct or allow the movement of the first and second protuberances, and the thickness W of the third protuberance is formed greater than the thickness V of the visor so that the inside portion of the third protuberance adjacent to the body of the helmet comes into frictional contact with the second protuberance of the sun visor placed between the body and the visor and the outside portion of the third protuberance comes into frictional contact with the first protuberance of the shield placed on the outside of the visor.

### BRIEF DESCRIPTION OF THE DRAWINGS

Other objects and aspects of the present invention will become apparent from the following description of embodiments with reference to the accompanying drawings in which:

FIG. 1 is an exploded perspective view of a shielding device for a helmet according to a first embodiment of the present invention;

FIG. 2 is a lateral view showing how the shield shown in FIG. 1 is being used;

FIG. 3 is a lateral view showing how the shield and sun visor shown in FIG. 1 are being operated simultaneously;

FIG. 4 is a sectional view of the hinge joining means shown in FIG. 1;

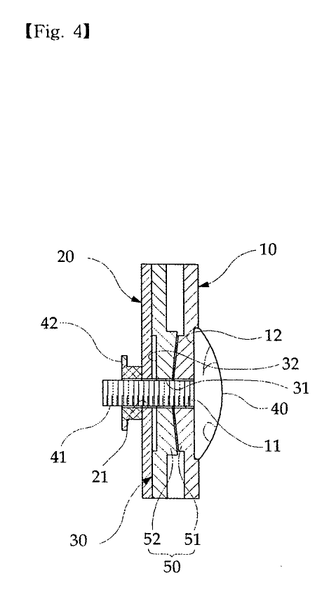

FIG. 5 is an exploded perspective view of a shielding device for a helmet according to a second embodiment of the present invention;

FIG. 6 is a drawing showing how the shield shown in FIG. 5 is being used;

FIG. 7 is a drawing showing how the shield and sun visor shown in FIG. 5 are being used;

FIG. 8 is a sectional view showing the configuration of the rotation interruption means shown in FIG. 5.

### DETAILED DESCRIPTION OF THE INVENTION

Below will be described in detail a shield device for helmet according to the embodiments of the present invention with reference to the accompanying drawings.

The helmet of the present invention comprises a shield 10, a sun visor 20 and a visor 30 as main components, and these are called collectively a shielding device in the present invention.

Of these, the shield 10 shuts out the driving wind coming into the portion of the face of the helmet wearer from ahead of the helmet during the driving of motorcycle, for example. The sun visor 20 placed on the inside of the shield 10 is made in such a size that can shield the upper half of the face of the helmet wearer so as to intercept dazzling due to sun light.

And, the visor 30 placed between the shield 10 and the sun visor 20 is for preventing rainwater from flowing into the helmet in case of rain, and is made in a shape of a short visor.

First Embodiment

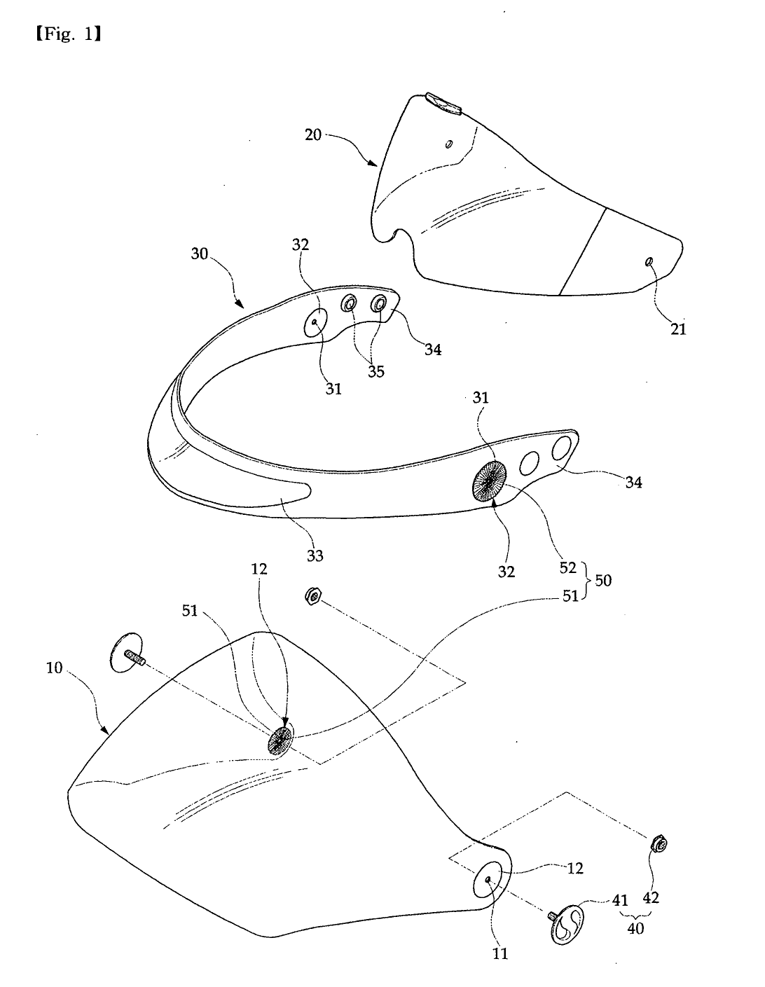

FIG. 1 is an exploded perspective view showing a shielding device for a helmet according to the first embodiment of the present invention.

In the first embodiment of the present invention, the shield 10, sun visor 20 and visor 30 are assembled together by a hinge joining means 40 in each end portion of both sides. The hinge joining means 40 is mounted on each of both sides of the helmet by screw joining such that the shield 10 and sun visor 30 can pivot up and down in one body or individually.

The hinge joining means 40 shown in FIG. 1 has, for example, first to third circular through holes 11, 21 and 31 formed at each end of both sides of the shield 10, sun visor 20 and visor 30. A fastening screw 41 passes through these through holes 11, 21 and 31, and screws with a nut 42.

According as the both end portions of the shield 10, sun visor 20 and visor 30 are tightened by the fastening screw 41 and nut 42 of the hinge joining means 40, the helmet wearer can pivot the shield 10 and the sun visor 20 simultaneously or individually.

Also, at wearer's convenience, the fastening screw 41 of the hinge joining means 40 can be unfastened to separate any one of the shield 10, sun visor 20 and visor 30 from the helmet or mount only one to the helmet optionally.

In addition to such a configuration, it can have a rotation interruption means 50 including a first toothed wheel 51 mounted on the inside of each end of the shield 10 and a second toothed wheel 52 mounted on the outside of each end of the visor 30.

Namely, since the first toothed wheel 51 are mounted on the inside of either end of the shield 10 and the second toothed wheel 52 which meshes with the first toothed wheel 51 are mounted also on the outside of either end, the shield 10 is pivoted intermittently according as the first and second toothed wheels 51 and 52 rotate, when the shield 10 is pivoted up and down.

Here, it is preferable that the fastening screw 41 of the hinge joining means 40 is installed passing through the rotation interruption means 50.

On the front of the visor 30 in contact with the inside of the shield 10 can be attached sealing members 33 in a belt form. The sealing members 33 is made of, for example, a rubber material, to prevent water leakage into the gap between the visor 30 and the shield 10.

And, the extended portion of the side end of the visor 30 can have snaps 35 for joining with the side portion of the helmet. By using the snaps 35, the assembly of the shield 10, sun visor 20 and visor 30 can be attached to or detached from the fixing members (not shown) formed on the helmet.

Next will be described the principle of operation of the shielding device according to the first embodiment.

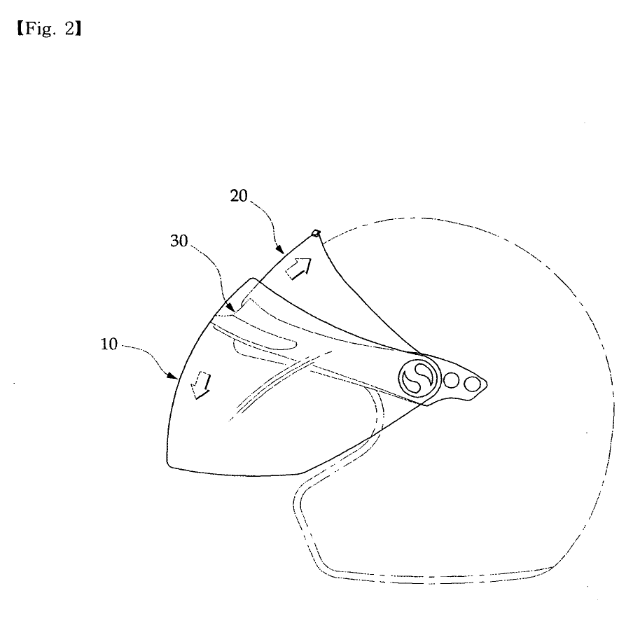

First, among the shield 10, sun visor 20 and visor 30, to use only the shield 10 without using the sun visor 20, the sun visor 20 is pivoted upward to position it in the top end portion of the shield 10, as shown in FIG. 2. Accordingly, the shield 10 is positioned ahead of the helmet so as to shield the face portion of the helmet wearer.

At this time, to adjust the angle of the shield 10 up or down, the shield 10 is pivoted slightly up or down. Namely, because the first and second toothed wheels 51 and 52 of the rotation interruption means 50 provided in the shield 10 and the visor 30 shown in FIG. 1 rotate in meshed state, the shield 10 and the visor 30 can be prevented from going down freely by self-weight.

Next, to use the shield 10 and sun visor 20 simultaneously, only the sun visor 20 is pivoted downward to position it in front of the field of vision of the helmet wearer.

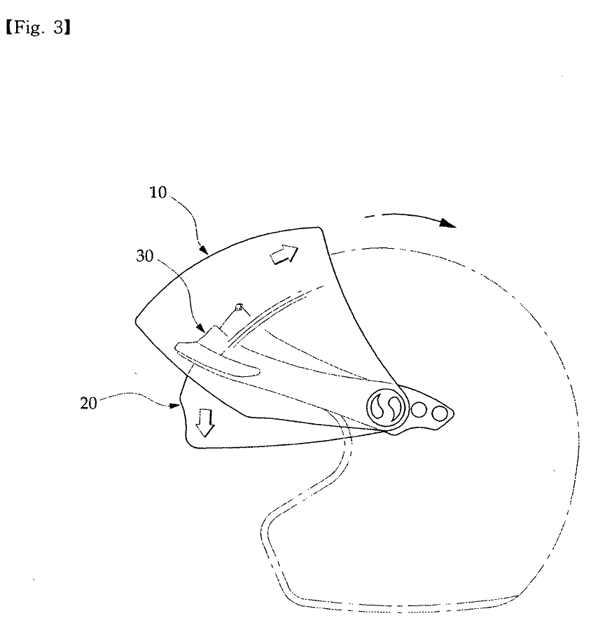

And, to use only the sun visor 20 without using the shield 10, only the shield 10 is pivoted upward with the sun visor 20 down to its original position, as shown in FIG. 3.

At this time, the pivoted shield 10 can have the angle adjusted properly. Namely, since the first and second toothed wheels 51 and 52 of the rotation interruption means 50 rotate in meshed state as described above, the helmet wearer can pivot the shield 10 at a desired angle to a stop, with the shield 10 interrupted from going down freely by self-weight.

Like this, it is possible to pivot the shield 10 and the sun visor 20 simultaneously in one body or individually one by one. Because both of the end portions of the shield 10, sun visor 20 and visor 30 are tightened each other by the fastening screw 41 of the hinge joining means 40, the helmet wearer can pivot by holding by hand the element he wants to pivot.

Meanwhile, to choose only the desired one out of the shield 10 and the sun visor 20 to mount it on the helmet, the fastening screw 41 of the hinge joining means 40 is unfastened to disassemble and then reassemble it.

Second Embodiment

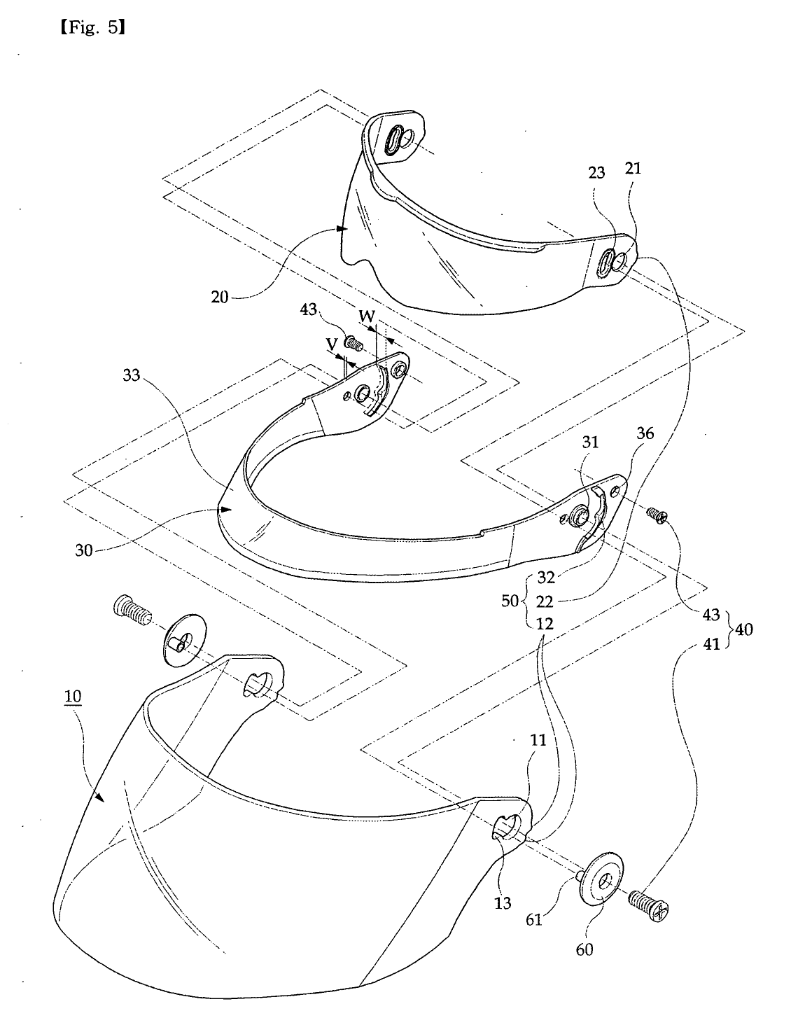

FIG. 5 is an exploded perspective view showing a shielding device according to the second embodiment of the present invention.

In the second embodiment, the configuration of the hinge joining means 40 and the rotation interruption means 50 is different from the first embodiment.

As shown in FIG. 5, a hinge joining means 40 exemplified in the second embodiment includes a first through hole 11 formed at either end portion of the shield 10, a third through hole 31 formed at either end portion of the visor 30, a second through hole 21 formed at either end portion of the sun visor 20, and a first screw 41 which passes through these through holes 11, 31 and 21 in sequence and screwed to the side of the helmet; and a second screw 43 which is fixed on the helmet body 1 through a fourth through hole 36 formed at a predetermined distance away from the third through hole 31 of the visor 30.

And, the rotation interruption means 50 of the hinge joining means 40, as shown in FIG. 5, includes a first protuberance 12 formed at either end of the shield 10, a second protuberance 22 formed at either end of the sun visor 20, and a third protuberance 32 which is formed around the third through hole 31 at either end potion of the visor 30 and has a predetermined elastic force. Here, the third protuberance 32 of the visor 30 formed in the direction of the circumference around the through hole 31 has self elastic force, so it obstructs the movement of the first and second protuberances 12 and 22.

When the third protuberance 32 formed at either end of the visor 30 is inserted into the slot provided on the first adjacent protuberances 12 and the second adjacent protuberances 22, respectively, the pivoting of the shield 10 and the sun visor 20 is obstructed by the third protuberance 32. On the other hand, if the wearer moves by hand the shield 10 or the sun visor 20 up or down, the first and second protuberances 12 and 22 press the third protuberance 32 to release the caught condition. Accordingly, the wearer can move the shield 10 or the sun visor 20 at a desired angle. When the movement of the shield 10 or the sun visor 20 is completed, the third protuberance 32 is restored to the original form by self elastic force, while it is inserted into the slot provided on the first adjacent protuberances 12 and the second adjacent protuberances 22, respectively, to obstruct the pivoting by self weight of the shield 10 and the sun visor 20.

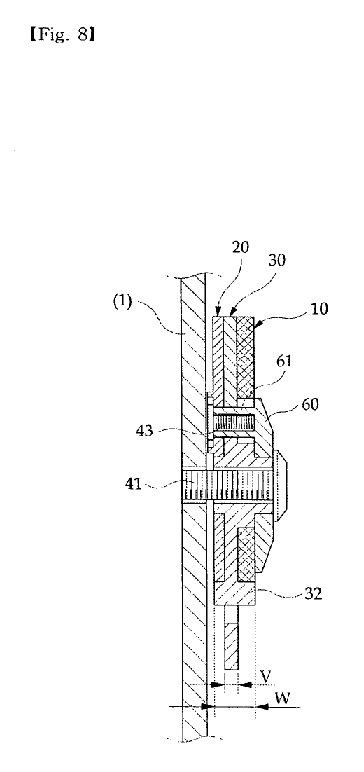

Here, the thickness W of the third protuberance 32 is formed greater than the thickness V of the visor 30. Therefore, as shown in FIG. 8, the inside portion of the third protuberance 32 adjacent to the helmet body 1 comes into frictional contact with the second protuberance 22 of the sun visor 20 placed between the body 1 and the visor 30, and the outside portion of the third protuberance 32 comes into frictional contact with the first protuberance 12 of the shield 10 placed on the outside of the visor 30.

As shown in FIG. 5, around the first and second protuberances 11 and 22 of the shield 10 and the sun visor 20 are formed the first and second long holes 13 and 23 into which a stopper 61 protruded in the head portion of a decoration washer 60 is inserted. Therefore, the shield 10 and the sun visor 20 have the pivot angle restricted within the length of these long holes by the stopper 61 inserted into the first and second long holes 13 and 23.

Operation of the shielding device in the second embodiment is almost identical with that in the first embodiment.

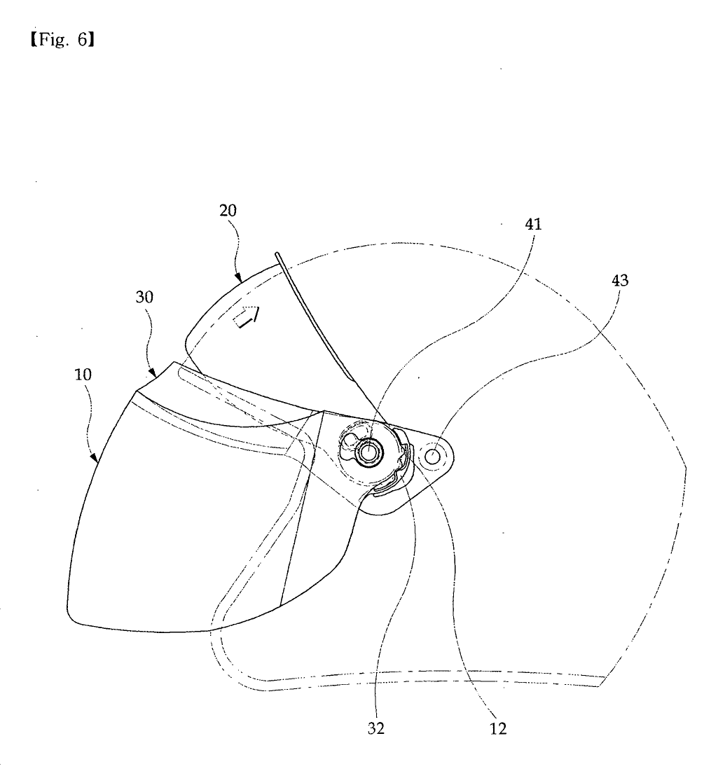

First, in the shield 10 and the sun visor 20, to use only the shield 10 without using the sun visor 20, as shown in FIG. 6, the sun visor 20 is pivoted upward to be turned up, then the sun visor 20 only is pivoted upward with the visor 30 fixed and it is positioned in the top end portion of the shield 10. At this time, the shield 10 may be positioned in front of the helmet to shield the face portion of the wearer. In this condition, the third protuberance 32 of the visor 30 is inserted and caught into the slot provided on the first adjacent protuberances 12 of either end of the shield 10 to obstruct its going down due to its self weight, so the shield 10 continues to maintain the condition.

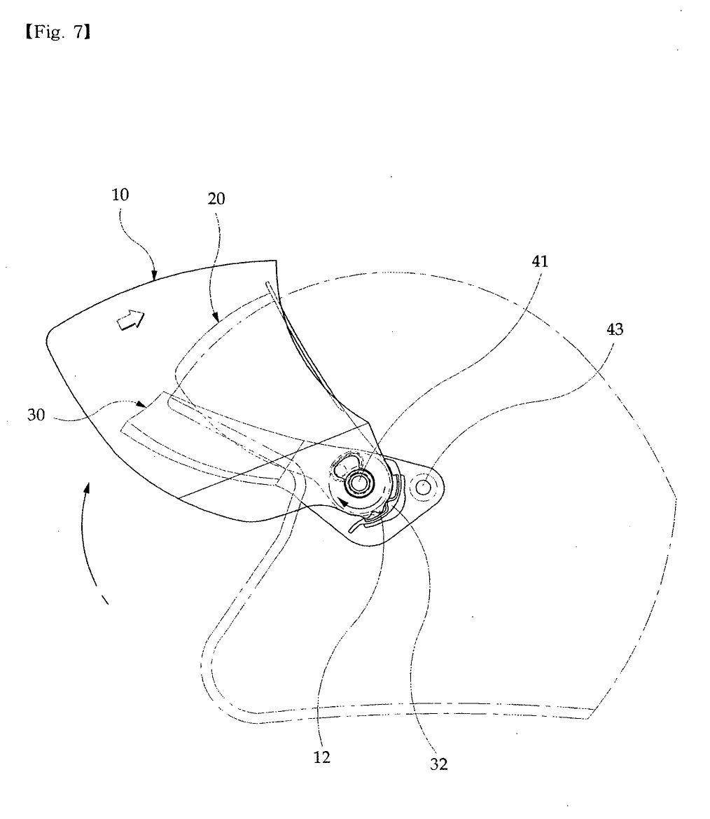

Next, when the shield 10 and the sun visor 20 are not used simultaneously, as shown in FIG. 7, both the shield 10 and the sun visor 20 are pivoted upward so that they are not positioned in front the field of vision of the helmet wearer.

And, to use only the sun visor 20 without using the shield 10, only the sun visor 10 is pivoted upward with the sun visor 20 put in a desired position.

The shield 10 and the sun visor 20, during the pivoting of each, have the pivot angle restricted within the length of the first and second long holes 13 and 23 by the stopper 61 of the decoration washer 60 inserted in the these long holes, so the shield 10 and the sun visor 20 are prevented from excessive pivoting.

Like this, the shield 10 and the sun visor 20 can be pivoted both in the same time or individually one by one. Namely, because both ends of the shield 10, the sun visor 20 or the visor 30 are joined together by the hinge joining means 40, the helmet wearer can pivot the desired element just by holding it by hand.

As described above, the shielding device according to the present invention has all of the shield, sun visor and visor with different functions each other which can be used optionally as necessary and can be easily attached and detached, so it can greatly improve the convenience of the helmet user.

Although the present invention has been described in connection with the exemplary embodiments illustrated in the drawings, it is only illustrative. It will be understood by those skilled in the art that various modifications and equivalents can be made to the present invention. Therefore, the true technical scope of the present invention should be defined by the appended claims.

1. A shielding device for a helmet comprising:
a shield to cover the opened portion of the helmet body;
a sun visor which is placed on the inside of said shield to shut off sunlight;
a visor which is placed between the shield and the sun visor to block the inflow of rainwater;
a hinge joining means which pivotably supports both ends of said shield and sun visor so that they can pivot up and down in one body and/or individually; and
a rotation interruption means which obstructs said shield, sun visor and visor from descending due to self weight as well as makes said shield be pivoted intermittently with respect to said visor.

2. The shielding device according to claim 1, further comprising snaps attached on the inside of the extended portion of both ends of the visor for joining with the helmet body.

3. The shielding device according to claim 1, wherein said hinge joining means includes a fastening screw which passes through first to third through holes in sequence formed at either end portion of the shield, sun visor and visor.

4. The shielding device according to claim 1, wherein said rotation interruption means includes a first toothed wheel mounted on the inside of either end of the shield, and a second toothed wheel mounted on the outside of either end of said visor.

5. The shielding device according to claim 1, further comprising belt-formed sealing members attached on the front of the visor in contact with the inside of the shield.

6. The shielding device according to claim 1, wherein said hinge joining means includes a first screw which passes through in sequence a first through hole formed at either end portion of said shield, a third through hole formed at either end portion of said visor and a second through hole formed at either end portion of said sun visor to screwed to the side of the helmet; and
a second screw which passes through a fourth through hole formed at a predetermined distance away from the third through hole of the visor to be fixed on the helmet body.

7. The shielding device according to claim 1, wherein said rotation interruption means includes a first protuberance formed at either end of the shield,
a second protuberance formed at either end of the sun visor, and
a third protuberance around the third through hole of either end portion of the visor which is inserted into a slot provided on the first adjacent protuberances and the second adjacent protuberances, respectively to support the rotation of these.

8. The shielding device according to claim 7, wherein the third protuberance of said visor has elastic force to obstruct or allow the movement of the first and second protuberances, and
the thickness of the third protuberance is formed greater than the thickness of the visor so that the inside portion of the third protuberance adjacent to the body of the helmet comes into frictional contact with the second protuberance of the sun visor placed between the body and the visor and the outside portion of the third protuberance comes into frictional contact with the first protuberance of the shield placed on the outside of the visor.

9. The shielding device according to claim 1, wherein first and second long holes are formed around the first and second through holes of said shield and sun visor, and
a stopper protruded in the head portion of the decoration washer is inserted into first and second long holes to restrict the pivot angle of said shield and sun visor within the length of these long holes.
